(19) United States
(12) Patent Application Publication
(10) Pub. No.: US 2004/0067585 A1
(43) Pub. Date: Apr. 8, 2004

(54) Cell cultivation surface and method of making the same

(76) Inventors: Yu-Chi Wang, (US); King-Ming Chang, (US)

Correspondence Address: FROMMER LAWRENCE & HAUG, 745 FIFTH AVENUE- 10TH FL., NEW YORK, NY, 10151, US

(21) Appl. No.: 10/371,738

(22) Filed: Feb. 21, 2003

(30) Foreign Application Priority Data
Oct. 7, 2002 (TW) 91123132

(51) Int. Cl. C12N 5/02; C12N 5/00

(52) U.S. Cl. 435/395

(57) ABSTRACT

The present invention claims and discloses a growth surface for cell and/or tissue culturing and its method of making thereof. More specifically, the instant invention claims and discloses a novel growth surface for culturing cells that maximizes growth surface, cell attachment, even distribution of nutrients and air to cells and providing a second chance for the unattached and/or dislodged cells for re-attachment by physically manipulating the growth surface into a geometric configuration, while compatible to be used in conjunction with any conventional cultivating system. The growth surface of the present invention can optionally have trimmed or folded ends to increase fluid movement thus increasing production of cellular product.

### RELATED APPLICATIONS/PATENTS & INCORPORATION BY REFERENCE

This application claims priority to Taiwanese patent application serial number 91123132 filed Oct. 7, 2002 entitled “A Carrier Used in Cell Cultivation and Its Manufacturing Process”. Reference is made to U.S. Application Serial No. 60/352,542 filed Jan. 31, 2002 entitled “Cell-Cultivating Device” and U.S. application Ser. No. 10/245,254 filed Sep. 16, 2002 entitled “Cell-Cultivating Device” and U.S. Publication No. U.S. 2002/0064875 A1 published May 30, 2002, incorporated herein by reference, together with any documents therein cited and any documents cited or referenced in their cited documents. Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited, are hereby expressly incorporated herein by reference. More generally, documents or references cited in this text, and, each of these documents or references (“herein-cited references”), as well as each document or reference cited in each of the herein-cited references (including but not limited to any manufacturer's specifications and instructions), are hereby expressly incorporated herein by reference.

### FIELD OF THE INVENTION

With the rapid developments of biotechnology, any cell culturing whether for prokaryotic cells or eukaryotic cells technology is becoming increasingly important and valued. Generally, eukaryotic cells are slow growing and vulnerable to injuries caused by shearing stress caused by aeration and feeding and contamination. Additionally these cell cultures are costly and difficult to maintain. Majority of the eukaryotic cells are anchorage-dependent and require a growth surface for them to grow and therefore cannot grow in suspensions. In order to remedy the shortcomings of the eukaryotic cell cultures, various carriers with growth surfaces were developed. The carriers currently known in the art generally have limited surface areas and cannot provide continuous even distribution of nutrients and oxygen to cells nor can these carriers provide reattachment of the dislodged cells. In addition, the carriers currently known in the art cannot protect cells from shear stress caused by the influx of cell medium and air into a culture vessel or bioreactor. Moreover, the materials that make up the growth surfaces also play a role in cell mortality and well-being. Currently available microcarriers generally contain cytodex 1, 2 and 3, porous matrix polyurethane, polyethylene terephthalate, and semi-permeable membrane such as polysulfone.

There are two types of carriers, particulate smooth surface carriers (nonporous or poreless) and porous carriers for anchorage dependent cells. Cells plated on the nonporous smooth surface carriers generally have a higher mortality rate because they cannot withstand the direct shear stress created by the movement of the growth medium and/or the influx of air. Additionally, the smooth surface does not lend itself to a large growth surface area, thus, limits the number of cells to be plated and/or adhered thus reduces protein production. The porous carrier on the other hand provides at least one three-dimensional cavity to house cells. In turn, these cells are protected from coming into direct contact with the shearing stress created by the mixing of the growth medium and aeration of the culture. The porousness of the carriers also creates additional surface areas for cell anchorage that protects cells from being in direct contact with shear stress created from aeration and feeding and thus increases cell density and leads to a higher protein production. In addition, the porous nature of the carriers lends itself to a stronger anchorage of the cells and also allows cell sub-culturing.

More often than not the cell-containing carriers must be disposed within a contained environment with constant supply of nutrient and oxygen such as a culture flask and/or a bioreactor. In order to accelerate cell growth and maximize protein production, stirring and/or aeration are necessary. The stirring and the aeration generate shearing force and create bubbles that causes cells dislodging from the carriers and ultimately cell death.

The present invention teaches generally a cell-cultivating growth surface having significantly improved efficiency in cell culturing and hence protein production. More specifically, the invention teaches a novel growth surface and its method of manufacturing same. The present invention claims and discloses a novel growth surface for culturing cells that maximizes cell attachment, increases cell density in a significantly increased surface area by geometric manipulation and facilitates even distribution of nutrients and oxygen to cells with minimum cell mortality ergo increases the production of cellular products.

The present invention also provides an increased cell cultivating growth surface carrier system that can be stacked on top of each other without overlapping to provide at least one three-dimensional space to facilitate the free flowing of the culture medium within the cultivation vessel or bioreactor and aeration without using any stirring mechanism and/or device. In addition, the unique geometric configuration of the growth surface maximizes growth medium movement and provides equal distribution of air to all cells with minimum cell mortality. Thus, the cell cultivating growth surface in accordance with the present invention remedies the shortcomings and the deficiencies in the existing technology.

### BACKGROUND OF THE INVENTION

Revolutionary advances in biotechnology and genetic engineering have created high demand to market cellular products, such as protein pharmaceuticals, cytokines, interferon, monoclonal antibodies, hormones, growth factors, insulin, viral products, vaccines, nucleic acids, enzymes, and cells and/or tissues for transplantation. The demand for these products has grown exponentially and will continue to do so thus, creating an ever-increasing need for efficient methods of producing industrial quantities of cell-derived products within the shortest time with the least contamination and requires the least space to cultivate the cells of interest. Efficient and cost-effective production of these products require a large-scale cell culture system capable of producing large quantity of cell-derived products through the most economic way at the minimum amount of time.

Both prokaryotic or eukaryotic cells can produce cellular products of desire. However, eukaryotic cells such as mammalian cells have become vital in providing high quality and quantity efficacious protein cellular products. Culturing mammalian cells has long been used to produce vaccines, genetically engineered proteins, pharmaceuticals and other cellular products. Generally, eukaryotic cells can be anchorage-dependent, anchorage independent or both. However, eukaryotic cells are generally anchorage-dependent, thus requiring a growth surface to anchor, mature and produce desired cellular products. Examples of anchorage-dependent cells are fibroblasts, epithelial cells and endothelial cells. Eukaryotic cells such as lymphocytes and some cancer cells are “anchorage-independent” cells and can grow in suspension. Regardless of its type, most eukaryotic cells in culture have the following characteristics in common and these characteristics play a key role in designing an efficient growth surface and cultivating device. Eukaryotic cells more often than not have a slow growth rate, are highly vulnerable to shearing stress and bubbles created by aeration and feeding and are susceptible to microbial contamination during cultivation.

The attachment of anchorage-dependent cells to a growth surface is the key to cell vitality and fundamental to all types of culture techniques including but not limited to traditional monolayer culturing or culturing with a carrier and/or microcarrier system. Since the proliferation of anchorage-dependent cells can only occur after adhesion to a suitable growth surface, it is important to use surfaces and culture procedures which enhance and maximize cell adhesion. Cell adhesion involves adsorption of attachment factors, such as proteins to a cultivation surface, contacting the cells with the cultivating growth surface, attaching the cells to a treated surface suitable for cell adhesion, spreading and replicating the adhered or attached cells across the growth surface until these cells come into contact with another surface-growing cell (i.e., “contact inhibition”).

In order to have a viable anchorage-dependent cell culture, the culture needs an appropriate cultivating growth surface or carrier, a mechanism for circulating culture medium particular to the cell type to be cultured and proper aeration with an adequate supply of gas to support and maintain cell growth. There are several different ways to culture cells and they are batch system in which nutrients are not replenished during cultivation although oxygen is added as required, fed batch systems in which nutrient and oxygen are monitored and replenished as necessary and perfusion systems in which nutrient and waste products are monitored and controlled.

There are several types of cultivation carriers that are currently known in the art. For example, dextran-based (e.g., Cytodex I, DEAE-dextran and Cytodex III, porcine collagen-coated dextran; Amersham-Pharmacia, UK) or coated polystyrene-based (e.g., SoloHill, U.S.) microcarrier. Microcarriers are typically very small and have diameters of approximately 50 to 250 micrometers, although larger or smaller sizes of microcarriers have been used (U.S. Pat. No. 5,114,855 issued May 19, 1992 to Hu et al.). A second type of cell-cultivation carrier includes a porous matrix material made from ceramics, polyurethane foam, or polyethylene terephthalate (PET). A third type of cell-cultivation carrier are semi-permeable membranes, such as polysulfone-based membranes.

Cell cultivation carriers can also be categorized according to its surface property. For example, there are non-porous or poreless and porous carriers. The porous carriers are generally more advantageous than the non-porous carriers since the porous carrier provides a greater surface-to-volume ratio as well as protection to insulate cells. Because of its porous nature, these carriers form multiple three-dimensional cavities within the growth surfaces and thus maximizes cell attachment and also protect cells from being dislodged and/or damaged from shearing stress resulted from aeration and growth medium circulation caused by stirring and impact during feeding and/or harvesting processes.

Many cell-cultivating systems currently available in the art employs microcarriers that are either porous and/or nonporous or poreless. These microcarriers such as microcarrier beads currently available are used in anchorage-dependent cell production systems. These microcarriers must be used in conjunction with a support equipment having stirring and/or aeration capability. However, a common problem with microcarrier systems is that the stirring action required to sustain the cell culture can damage or even kill the cells thereby decreasing the efficiency of the cultivation system and the production of the desired cellular product.

Microcarrier systems can also be fabricated in small sphere from an ion exchange gel, dextran, polystyrene, polyacrylamide, or collagen-based material. These materials have been selected for their compatibility with cells, resilience to agitation and specific gravities that can maintain the microcarriers suspended in growth media. Microcarriers are generally kept in a growth medium suspension with gentle stirring disposed within a vessel in order to ensure equal distribution of nutrients and air to all cells. Microcarrier system is currently considered to be the most suitable system for large-scale cell culture because it has the highest surface to volume ratio and enables even distribution of nutrients to cells.

Nevertheless, current microcarrier culture system has serious disadvantages. These disadvantages include high costs and high cell mortality rates due to exposures to high levels of shearing forces caused by stirring and aeration during cultivation. Most commonly used microcarriers utilize porous non-rigid dextran as a support matrix. This compressible matrix is thought to reduce potential damages to the microcarriers and their attached cells when the microcarriers collide in agitated reactors (Microcarrier Cell Culture: Principles and Methods, Pharmacia Fine Chemicals, Uppsala, Sweden, pages 5-33 (1981)). These porous microcarriers however, also have serious disadvantage in retaining cellular products that results in the adsorption of growth factors and other components from the medium (Butler, M., “Growth Limitations in Microcarder Cultures”, Adv. Biochem. Eng./Biotech. 4:57-84 (1987)). Other microcarrier materials have disadvantages as well. For example, polystyrene microcarriers have an unacceptably low rate of cell attachment.

Other types of carriers known in the art also have disadvantages. For example, U.S. Pat. No. 5,266,476 issued Nov. 30, 1993 to Sussman et al. relates to a two-layered carrier for cell cultivation with one layer made of a non-woven fabric and the other layer being a polymer net. The carrier advantageously provides a thin-layered surface to facilitate the supply of nutrients and oxygen to the cells. This type of carrier further provides an improved surface/volume ratio thus, promoting higher cell density. However, in order to increase the structural integrity of the microcarrier, a polymer composite was used. The polymer composite occupies surface areas of the microcarrier thus severely limit the growth surfaces for cells. In addition, the rigidity of the microcarrier can cause harsh contacts with the growth surface during stirring and increase cell mortality that ultimately leads to reduction in cellular products.

In another example, U.S. 2002/0064875A1, published May 30, 2002 reveals a carrier for cell attachment and growth. The carrier is made from a non-woven structure having an activated surface with cell affinity. This carrier is manufactured by forming a non-woven structure from extruding a melted polymer from a nozzle. The surface of the non-woven fabric carrier is pleated or created unevenly by using a hot pressing process to increase its structural integrity. Although the fabric shape of the carrier can provide an environment for cell attachment and growth these carriers are still two-dimensional flat strips. When there are more than one strip placed in a cultivating system, the microcarriers in the flat strip may overlap each other thus causing cell death. The overlapping of the strips, thus the carriers can cause cell death or at the minimum, unequal distribution of nutrients and air to cells resulting in cell mortality and decreased cellular production.

U.S. Pat. No. 4,266,032 issued May 5, 1981 to Miller et al. relates to a method of submerging cells by using a microcarrier support having cross-linked polystyrene resin beads derivatized with amino acids, peptides, or hydroxy carboxylic acids.

U.S. Pat. No. 4,789,634 issued Dec. 6, 1988 to Müller-Lierheim et al. relates to a carrier for cultivating animal and/or human cells in a fermenter. The carrier has a pressure-resistant matrix formed from a polymer and a capillary system for carrying the liquid cell culture medium therethrough without any cell growth occurring. The carrier has interstices for three-dimensional cell growth, growth factors for the cells being covalently bonded to the boundary surfaces of the interstices. The carrier provides a high cell density in the order of 10⁹ cells/ml, by three-dimensional cell growth.

U.S. Pat. No. 5,015,576 issued May 14, 1991 to Nilsson et al. relates to making particles which enclose cavities by adding a water-insoluble solid, liquid or gaseous cavity generating compound to an aqueous solution of matrix material. Subsequent to forming particles by dispersion in a water-insoluble dispersion medium, the matrix is rendered insoluble in water by cooling, covalent cross-linking or by polymerization. The cavity generating compound is washed out, whereafter the particles can be used as ion exchangers in gel filtration processes, in hydrophobic chromatography or in affinity chromatography, optionally subsequent to derivatizing the particles. The particles can also be used as microcarriers in cultivating anchorage-dependent cells.

U.S. Pat. No. 5,385,836 issued Jan. 31, 1995 to Kimura et al. relates to a carrier for animal cells attachment during cell culturing or for immobilization of animal cells. This carrier is produced by coating a porous substrate with a cell adhesive material in the form of a mixture containing chitosan. The porous substrate is a nonwoven fabric prepared by impregnating a nonwoven fabric web with a binder resin which contains silk fibroin, gelatin and chitosan. Coating is carried out by contacting the nonwoven fabric with a solution prepared by adding silk fibroin and gelatin to an acidic aqueous solution of chitosan to coat the nonwoven fabric, drying the coated nonwoven fabric and treating the dried nonwoven fabric with an alkali to render the chitosan insoluble.

U.S. Pat. No. 5,565,361 issued Oct. 15, 1996 to Mutsakis et al. relates to a bioreactor having a motionless mixing element with attached cells method for the enhanced cultivation and propagation of cells in a bioreactor. The bioreactor has a housing and a motionless mixing element, the attachment of cells to the mixing element and a nutrient composition permitting attached cells to grow and divide. The motionless mixing element and the bioreactor have a porous, fibrous sheet material such as a corrugated or knitted woven wire material, such as stainless steel or titanium, and predetermined dimensions for the height and diameter of the fiber in order to provide a maximum surface area for the attachment of the cells to be cultivated.

U.S. Pat. No. 5,739,021 issued Apr. 14, 1998 to Katinger et al. relates to a porous carrier for biocatalysts with a water-insoluble inorganic filler and a polyolefine binder selected from polyethylene and polypropylene, has open pores to allow cells to penetrate and grow within its pores. The density is above 1 g/cm³.

U.S. Pat. No. 6,001,642 issued Dec. 14, 1999 to Tsao relates to a bioreactor and cell culturing method using the bioreactor. The bioreactor contains a dome-shaped culture vessel with walls defining an interior volume, an apex, a bottom circular edge, and an axis of symmetry perpendicular to the bottom circular edge; and a gas-permeable membrane fluidtightly integrated with the bottom circular edge of the culture vessel. The bioreactor may further contain a rotating base fluidtightly integrated with the gas-permeable membrane wherein the rotating base is fixed to rotate about the axis of symmetry of the dome-shaped culture vessel set on a substantially horizontal axis.

U.S. Pat. No. 6,214,618 issued Apr. 10, 2001 to Hillegas et al. relates to a method of making microcarrier beads by forming a bead made of a lightly crosslinked styrene copolymer core with functional groups on the surface of the bead and washing the microcarrier beads with basic and acidic solutions to make the beads compatible for cell culture. The microcarrier bead can also be made of a styrene copolymer core with a tri-methylamine exterior which has been washed in basic and acidic solutions to make the beads compatible for cell culture.

U.S. Pat. No. 6,358,532 issued Mar. 19, 2002 to Starling et al. relates to calcium phosphate-based (CaP) microcarriers and their use in cell culturing systems, chromatography and implantable biomedical materials.

Notwithstanding the variety of carriers taught in the foregoing art, in view of both the great importance of cell cultivation processes and the deficiencies of the carrier systems currently known in the art, the present invention teaches and claims a cell cultivating system and device with a high oxygenation capacity, significantly-increased growth surface areas, cavities that shield and protect cells from damages caused by shearing forces generated by aeration and feeding. The device and the system according to the present invention also reduces contamination and increases cellular production.

### SUMMARY OF THE INVENTION

The present invention provides an efficient and novel growth surface suitable for culturing cells and its method of manufacture thereof. More specifically, the present invention teaches and discloses a novel cell cultivation growth surface and/or a carrier for efficiently facilitating adhesion and growth for prokaryotic and eukaryotic cells and in turn increase production of cellular products. Moreover, the instant invention claims and discloses a novel carrier or a growth surface or a cell cultivating system for culturing cells that maximizes cell attachment, encourages cell re-attachment for dislodged cells, increases surface area for cell growth, cell density, multicellular aggregates and evenly distributes nutrients and air to cells without using any stirring and/or mixing mechanism. The present invention also provides a cell cultivation carrier system or a growth surface for cells that generate significant cellular proliferation thus increasing the production of cellular products.

The objects of the present invention include but are not limited to: providing a novel cell cultivating growth surface in any size, shape, form, structure, geometric configuration from any suitable material and any suitable coating material that significantly increases the efficiency of the cell cultivating process; providing a novel cell cultivating growth surface, pellet, strip, sheet, carrier or microcarriers that maximizes cell adhesion and proliferation by increasing growth surfaces and provide constant aeration and feeding without causing any trauma or injury to the cells; providing a novel cell cultivating carrier that can also serve as a static mixer without employing a conventional mixing mechanism; providing a novel cell cultivating carrier that evenly distributes oxygen and nutrients to all cells without injuring or killing the cells; providing a cell cultivating surface carrier with enhanced mixing and homogenization of the culture medium; providing a cell cultivation carrier with a greater growth surface that is compatible in size with any conventional cell cultivating device such as a bioreactor or a flask; providing a growth surface for cells that can overlap in space due to its unique geometric configuration without killing any cells or hindering the movement of air and nutrients by geometrically manipulating the grow surface which resulting in a substantially increased growth surface area and for equal distribution of air and nutrients to all cells and cell adhesion; providing a growth surface for eukaryotic and prokaryotic cells that is formed from a flat surface that is folded into an advantageous geometric shape thus creating additional growth surface in order to maximize cell attachment, cell growth and cell proliferation; providing a cell cultivation carrier that is a flat surface with at least one end of the surface being trimmed into a non-right-angle-cut in order to create a turbulence with the culture medium as the medium travels through the cut thus increasing the mixing rate of the culture medium resulting in cells receiving nutrients and air more frequently thus promotes cell proliferation and increases cellular production; providing a growth surface in the shape of a pellet; providing a cultivating system or a growth surface with a hydrophillic coating in order to facilitate cell adhesion, cell growth, cell proliferation and cellular production; providing a method of making a substantially larger and sturdier growth surface without physically increasing the dimension of the growth surface and thus the cell cultivating device to accommodate the increased growth surface area.

In a first embodiment of the present invention, a two-dimensional growth surface made from flexible and porous material is disclosed to enhance cell attachment, promote cell growth and increase cellular production.

In a second embodiment of the present invention, the growth surface is flexible and can be folded and/or physically manipulated and/or constructed in any geometric shape suitable for any cultivating system such as a flask or a bioreactor. The growth surface according to the present invention can be formed in any desired shape, preferably creating a three-dimensional cavity such as W-shaped, V-shaped, ribbon-shaped, spiral-shaped, bowl-shaped, boat-shaped, shovel-shaped, or dust-pan shaped growth surface. These three-dimensional cavities can encourage cell attachment and also provide a shelter to protect the cells from coming into direct contact with an influx of air during aeration and the movement of the nutrients during feeding.

In a third embodiment of the present invention, the growth surface is shaped as a pellet.

In a fourth embodiment of the present invention, the growth surface is shaped as a carrier.

In a fifth embodiment of the present invention, the growth surface is ribbon-like.

In a sixth embodiment of the present invention, the growth surface has a first end and a second end and is made from a flexible yet rigid porous material. The growth surface can be deformed, bent, pleated, creased or folded in any shape either manually or mechanically to create at least one three-dimensional structure to house cells. The first end and second end of the growth surface can also be trimmed in any angle other than a right angle and the first end and the second end may also be bent or folded such that when the culture medium flows through the growth surface and come to the end of the growth surface the medium, because of the configuration at the end of the growth surface will create a current and/or a turbulence that further facilitates the mixing of the medium and an opportunity for reattachment of cells to the growth surface that failed to do so during the initial plating. The folding and/or bending and/or pleating and/or deformation of the growth surface not only increases surface area to promote cell growth but also adds to the structural integrity of the growth surface and reduces cell mortality since cells are no longer directly exposed to the shearing stress created by aeration and feeding.

The growth surface according to the present invention not only provides more areas for cell attachment, opportunities for cell re-attachment, it can also be in any shape and any dimension made from any suitable material for cell culturing such as polyamide, polyester, polyurethane, polyaramid, fluorocarbon polymers, polyvinyl alcohol, polyethylene terephthalate, polypropylene, high-density polyethylene, and polyethylene with any suitable coating and because the surface can be folded in order to increase surface areas, it can be of any size in order to accommodate and be compatible with any conventional cell cultivating device such as a flask or a bioreactor. Additionally, the current and/or the turbulence resulting from the culture medium travelling through the novel growth surface of the present invention provide additional opportunities for the loose unattached cells to re-attach themselves to the growth surface to proliferate and produce desired cellular products.

In yet another embodiment, the present invention teaches a method of manufacturing a growth surface for eukaryotic and/or prokaryotic cells comprising the steps of treating a surface to encourage cell attachment, providing a flat surface, folding the flat surface in any geometric shape that maximizes surface area, providing cell attachment, promoting cell growth and proliferation and disposing the growth surface in any conventional cell cultivating device.

The novel growth surface in accordance with the present invention can be made from any material that is flexible, yet sturdy and capable of maintaining any configuration given. The novel growth surface can be made from any polymer such as polypropylene and/or polyethylene or resins. The growth surface can also be made from a non-woven fabric made from interconnected pieces of polyethylene terephthalate (PET) or a sheath-core fabric, such as CELBOND® (KoSa, Houston, Tex.), which can be composed of polyethylene-polyethylene terephthalate (PE-PET), copolyethylene-polyethylene terephthalate (CoPE-PET), or polypropylene-polyethylene terephthalate (PP-PET), respectively, and treated with any chemical and biological material that can make the surface hydrophilic. Preferably in the sheath-core fabric, the sheath component comprises high-density polyethylene (HDPE) and the core component comprises polyethylene terephthalate (PET). Surface treatment is optionally conducted in the sheath layer. The deforming, folding, bending and pleating of the novel growth surface can be accomplished either manually or by any conventional device.

These and other embodiments are disclosed or are obvious from and encompassed by the following Detailed Description.

### BRIEF DESCRIPTION OF THE DRAWINGS

The following Description, given by way of example, is not intended to limit the present invention to any specific embodiments described. The Description may be understood in conjunction with the accompanying Figures, incorporated herein by reference.

FIG. 1(a) shows a novel growth surface of the first embodiment of the present invention wherein the growth surface is three-dimensional V-shaped with four growth surfaces.

FIG. 1(b) shows a novel growth surface of the first embodiment of the present invention wherein the V-shaped growth surface has at least one non-right-angle-shaped turning point disposed at the first end and/or the second end of the growth surface.

FIG. 2(a) shows a novel growth surface of the second embodiment of the present invention wherein the growth surface is a three-dimensional U-shaped surface.

FIG. 2(b) shows a novel growth surface of the second embodiment of the present invention wherein the U-shaped growth surface has at least one non-right-angle-shaped turning point disposed at the first end and/or the second end of the growth surface.

FIG. 3(a) shows a novel growth surface of the third embodiment of the present invention wherein the growth surface is three-dimensional W-shaped surface.

FIG. 3(b) shows a novel growth surface of the third embodiment of the present invention wherein the W-shaped growth surface has at least one non-right-angle-shaped turning point disposed at the first end and/or the second end of the growth surface.

FIG. 4(a) shows a novel growth surface of the fourth embodiment of the present invention wherein the growth surface is in a shape of a ribbon.

FIG. 4(b) shows a novel growth surface of the fourth embodiment of the present invention wherein the growth surface is a V or U-shaped ribbon having a three-dimensional structure.

FIG. 5(a) shows a novel growth surface of the fifth embodiment of the present invention wherein the growth surface is spiral shaped.

FIG. 5(b) shows a novel growth surface of the fifth embodiment of the present invention wherein the growth surface is a three-dimensional spiral structure.

FIG. 6 shows a novel growth surface of the sixth embodiment of the present invention wherein the growth surface is bowl-shaped.

FIG. 7 shows a novel growth surface of the seventh embodiment of the present invention wherein the growth surface is boat-shaped.

FIG. 8 shows a novel growth surface of the eighth embodiment of the present invention wherein the growth surface is shovel-shaped.

FIG. 9 shows a comparison for an overall glucose consumption rate per hour over time between cells that are attached to the growth surface of FIG. 1(a) and the conventional FIBRA-CEL® (manufactured by Bibby Sterilin, England and distributed by New Brunswick Scientific, Inc., Edison, N.J.) disk-shaped carriers.

FIG. 10 shows a comparison for an overall glucose consumption rate per hour over time between cells adhered to the growth surface shown in FIG. 1(b) and cells adhered to a growth surface without any three-dimensional structure.

These and other embodiments will be described and/or will be obvious from the following detailed description.

### DETAILED DESCRIPTION

The following detailed description, given by way of example, is not intended to limit the invention to any specific embodiments described. The detailed description may be understood in conjunction with the accompanying figures, incorporated herein by reference. Without wishing to unnecessarily limit the foregoing, the following shall disclose the present invention with respect to certain preferred embodiments. The embodiments in accordance with the present invention are suitable for prokaryotic and/or eukaryotic cell cultures and particularly for animal cells and/or mammalian cells. The present invention, inter alia, teaches a novel growth surface suitable for culturing any cells and/or microorganisms that can be colonized in vitro, have a high cell adhesive and/or plating rate with minimum cell mortality, within a shorter time to produce increased cellular products than cells plated on any conventional microcarriers by manipulating the shape of the novel growth substrate in accordance with the present invention.

The novel growth surface according to the present invention can be made from any material that is flexible, yet sturdy and capable of maintaining any configuration given. For example, any polymers, such as polyethylene terephthalate, polypropylene and/or polyethylene or resins. Preferably, the material and/or materials used to construct the instant invention will be porous, i.e., contain at least one pore and/or at least one cavity, although non-porous and/or poreless materials are also contemplated. The pores in accordance with the present invention provide a maximum surface area to facilitate cell attachment, cell adhesion and cell proliferation thereby providing a maximum cell density and thus, maximum cellular products.

The novel growth surface of the instant invention is preferably formed from a porous material, such as a non-woven fabric. Preferably, the non-woven carrier of the instant invention is made from interconnecting pieces of polyethylene terephthalate (PET) fabric or a sheath-core fabric, such as CELBOND®, which can be composed of polyethylene-polyethylene terephthalate (PE-PET), copolyethylene-polyethylene terephthalate (CoPE-PET), or polypropylene-polyethylene terephthalate (PP-PET), and treated with any chemical or biological material that can make the surface hydrophilic.

Non-woven fabrics are known to one of ordinary skill in the art. For example, a non-woven fabric which is adapted for needle piercing treatment can be used. The needle piercing treatment produces three-dimensional entanglement of the constituent fibers with each other so the cells are easily immobilized when enter into the nonwoven fabric and the efficiency of proliferation is improved. Further, the instant invention encompasses the use of a thick woven or knitted fabric with a three-dimensional woven structure or knitted structure. Furthermore, the invention encompasses the use of a sheath-core non-woven fabrics which are generally known in the art. The fiber density of the non-woven, woven or knitted fabric can be in a range of from 0.02 g/cm³ to 0.1 g/cm³. However, it is preferable that the non-woven, woven or knitted fabric is not constructed from a memory-retaining material, i.e., the non-woven, woven or knitted fabric of the instant invention does not resume its original shape upon deforming, bending, folding or creasing.

The non-woven fabric used to form the growth surface of the present invention can be prepared from known process for preparing a non-woven fabric such as spin-bonding, thermal bonding, calendering, or needling. In addition, a non-woven fabric may also be prepared by impregnating a web mainly comprising hydrophilic fibers with the binder resin and drying the impregnated web. Furthermore, the growth surface of the present invention can be formed using any suitable homogenous material or suitable heterologous materials.

The novel growth surface of the present invention can be in any size, shape, form, structure or geometric configuration so long as it is in accordance with the present invention. The growth surface of the instant invention can be in any suitable form, such as a pellet, a strip, a sheet, or any three-dimensional structure. In one embodiment, the growth surface of the present invention is in the form of a strip. The strip is flexible and can be two-dimensional or folded or constructed in any geometric shape to become three-dimensional that is adapted to maximize surface area, cell attachment and cell growth. The strips can be folded and/or physically manipulated and/or constructed in any geometric shape suitable for any cultivating system such as a flask or a bioreactor. The strip according to the present invention can be formed in any desired shape, preferably creating a three-dimensional cavity such as W-shaped, V-shaped, ribbon-shaped, spiral-shaped, bowl-shaped, boat-shaped, shovel-shaped, or dust-pan shaped growth surface. The folds can comprise bends, creases, pleats or any deformations of any type that creates at least one three-dimensional cavity to host cells and to protect and prevent cells from coming into direct contact with the influx of air and the movement of nutrients. Further, the folds, bends, creases, pleats or deformations facilitate the even and more frequent and rapid distribution of oxygen and movement of nutrients and redistribution of cells. These folds, bends, creases, pleats or deformations also prevent the overlapping of individual growth surfaces (when more than one surfaces are disposed within the flask and/or culturing device) and the creation of three-dimensional or branch-like channels to allow the free flow of the media.

In another embodiment of the invention, the growth surface has a first end and a second end. The first and second ends may optionally be trimmed in any angle and the ends may optionally bent or folded such that when the culture medium flows through these ends, a current and/or turbulence is created due to the configuration at the end of the growth surface. The current or turbulence further facilitates the mixing of the medium and re-attachment of the dislodged cells. The folding and/or bending and/or pleating and/or deforming of the growth surface not only increases surface area for cell attachment but it also adds to the structural integrity of the growth surface and reduces cell mortality since cells no longer come into direct contact with the air and medium.

The growth surface of the present invention may also be in the form of a pellet that can be of a variety of sizes having a diameter ranging from about 1 millimeter to about 250 millimeters, although any diameter may be deemed suitable depending on the individual needs. Preferably, the growth surface is in the form of an irregularly-shaped pellet that is loosely packed as a matrix in a culture tank or a culture flask or a bioreactor. The porous carrier or growth surface or pellet can form a loosely packed bed that allows for easy and efficient distribution of the cells during inoculation and assures maximum cell adhesion on the surfaces of the porous pellet or porous growth surface or porous carrier.

The growth surface of the present invention provides enhanced growth, vitality and viability of the cells in at least three ways.

First, when the growth surface is submerged in the growth medium due to its geometric configuring, there can be a gentle stirring occurring in the porous spaces of the growth surface. Thus, the porous growth surface can function as a static mixer without ever using a conventional mixer since it creates a gentle stirring environment upon movement of the growth medium. Without being bound by theory this gentle stirring effect improves the growth of the cells by gently mixing and distributing nutrients and redistributing dislodged cells and increasing the likelihood that the dislodged cells will and can re-adhere and/or re-attach to the growth surfaces. Redistribution of dislodged cells particularly with anchorage-dependent cells can help improve overall culture viability thus increase cellular production.

Second, when the growth surfaces is not submerged in the growth medium the porous carrier or the porous growth surfaces provides efficient oxygenation to cells while protecting the cells from being directly exposed to a gaseous environment. Thus, the porous carrier functions as an efficient oxygenator of the cells. Direct exposure of cells to a gaseous environment can be detrimental to cell growth particularly for animal cells and further cause damage or death. When the growth surface emerges from the culture medium, a portion or coating of growth medium remains on the surfaces of the porous carrier, thereby covering the cells embedded or growing on the growth surface with a thin layer of growth medium. This creates a thin gas-growth medium interface that allows for efficient diffusion of oxygen thus allowing the cells to efficiently uptake oxygen without directly exposing the cells to air hence avoiding the trauma by shearing stress. Furthermore, the porous carrier maximizes a surface area for cell growth and for cells to come into contact with an air-growth medium layer.

Third, the porous carrier or the porous growth surface functions as a depth filter to trap cells during plating and/or inoculation thereby increasing the number of adhered cells.

The ideal density for the growth surface or the carrier in accordance with the present invention may be in the range of 1.02 g/cc to 1.05 g/cc, although lighter or heavier material may be better suited for different applications for different types of cells. In addition to differences in surface chemistry carriers or growth surface made from one substance or another differs in such characteristics as rigidity, porosity and adsorptive capacity. Differences in handling characteristics, durability, shelf-life and ease of sterilization all distinguish one substance from another as does overall manufacturing costs. From the standpoint of commercial potential, the present invention is adaptable and suitable to all of these variables.

One of skill in the art will understand that certain characteristics of a growth surface can have an effect on its performance. Carrier or surface characteristics, such as surface properties, carrier density, size, optical properties, toxicity and rigidity can affect the performance of the growth surface and thus the performance of the cell culture particularly with respect to the cell density and the overall production of cellular products. Specifically, the size of the pores of the growth surfaces can affect the performance of the cells. Although one of ordinary skill in the art will appreciate that any growth surface pore size known will be suitable, the pore size is preferably in the range from 1 micrometer to 500 micrometers and preferably from 50 micrometers to 450 micrometers, more preferably from 50 micrometers to 200 micrometers. During cell cultivation, the cells attach and spread out throughout the surfaces of the porous carrier of the instant invention thereby allowing the production of very high cell densities.

The growth surface of the instant invention can have a variety of surface properties. Such properties can include rough, bumpy, irregular, coarse, jagged, toothed, textured, abraded, bent, pleated, folded, deformed or smooth surfaces. Further, any combination of the aforementioned properties and/or any additional suitable properties can comprise the growth surface. Preferably, the growth surface is porous. Further, the growth surface can comprise treating the surface with other substances, including for example, biological coatings (e.g., “cell adhesion factors”) including growth factors, selected protein, amino acids, collagen, antibodies, chemical materials, such as hydrophilic substances, and other such materials to facilitate and promote cell attachment, proliferation and spreading. It will be understood to one of ordinary skill in the art that a hydrophilic surface will help promote cell adhesion and attachment. Also, materials such as specific proteins, ligands, or antibodies, can be added to the growth surface to provide selective adhesion of different cell types or cell populations. Furthermore, the growth surface can be treated by plasma, corona, ultraviolet, radiation or wet type chemistry to improve cell affinity and/or attachment. It is also known in the art to graft functional groups onto the growth surface to enhance cell adhesion. Further, the cell cultivating carrier of the instant invention can also contain both a cell adhesion factor and a positively-charged molecule to improve cell attachment and stabilization during growth, for example as described in U.S. Pat. No. 5,512,474.

It will be appreciated that the cell adhesion to a surface is a multi-step process, comprising initial cell attachment characterized by weak binding and little cell shape change followed by cell spreading which produces stronger binding of cells to the growth surface. The initial attachment can be mediated by a non-specific mechanism such as a charged surface. In contrast to the initial attachment, cell spreading appears to require the presence of specific receptor-ligand interactions between cell surface receptors and certain cell adhesion factors, such as the glycoproteins fibronectin, laminin, and collagen. All three types of these glycoproteins have been purified and added to tissue culture surfaces to promote cell adhesion and cell growth. These glycoproteins must be adsorbed on the cell cultivation surface before they can promote cell attachment, proliferation and spreading. Studies have shown that a coating of gelatin or denatured collagen facilitates the attachment and growth of mammalian cells. Further details describing cell adhesion and spreading can be found in Microcarrier Cell Culture: Principles and Methods, Pharmacia Fine Chemicals, Uppsala, Sweden, pages 5-33 (1999).

Any type of cell can be plated and/or grown on the growth surface and methods in accordance with the instant invention. The embodiments of the present invention can be used to culture eukaryotic cells and/or prokaryotic cells. In one embodiment, the cells are preferably mammalian cells, more preferably human cells. The instant invention further contemplates both recombinant and non-recombinant prokaryotic or eukaryotic cells. Eukaryotic cells can include, for example, insect cells, e.g. Sf-9, primate cells, e.g., Vero, mouse, e.g., BHK or C-127, hamster, e.g., CHO, fungal, e.g., Saccharomyces or Scizosaccharomyces, human, e.g., tumor, osteoblast and mesenchymal stem cells, or monkey cells. Prokaryotic cells can be any aerobic or anaerobic, Gram positive or Gram negative bacteria, or recombinant or non-recombinant, including, but not limited to, Escherichia coli and Bacillus subtilis. In one embodiment, the cells used in accordance with the present invention are anchorage dependent eukaryotic cells.

The growth surfaces of the present invention can also be shaped and/or sized to be used with any suitable bioreactor (i.e., “cell culture system” or “culture vessel”) configuration known in art. In other words, the invention is compatible with any bioreator with any shape, size or dimension. For example, suitable bioreactors can be in the form of tubes, microtiter wells, columns, hollow fibers, roller bottles, plates, dishes, hollow fibers, large-scale bioreactors, small-scale bioreactors, multi-chamber reactors, or reactors with bellows or other like means capable of moving medium from one chamber to another. Small-scale bioreactors, such as laboratory-scale systems are generally less than approximately 5 liters. Large scale bioreactors are generally in the range of approximately 5 to several hundered liters and must be maintained in specially designed vessels allowing monitoring and control of parameters such as gas tensions and pH.

It will be appreciated that in order to successfully create a healthy cell culture in a bioreactor, certain minimum requirements must be met. For example, an aeration system to bring the correct amount of oxygen to the cells without causing shear damage and to regulate the pH of the environment, surfaces for supporting anchorage-dependent cells, and means to enable operators to sample and monitor the contents of the bioreactor without contaminating the culture.

The growth surfaces of the instant invention can be compatible with spinner and rod-stirred bioreactors. The spinner vessel is well known in the art of suspension culture for anchorage-independent cells. The culture can be stirred by a suspended teflon-coated bar magnet which is driven by a magnetic stirring base unit. Spinner vessels can be obtained commercially for example from Wheaton Scientific (Milville, N.J.) and Bellco Glass Inc. (Vineland, N.J.). More discussion on the operation of spinner and rod-stirred bioreactors can be found in Microcarrier Cell Culture: Principles and Methods, Pharmacia Fine Chemicals, Uppsala, Sweden, (1999).

The cell cultivating carrier of the instant invention can also be used with air-lift and/or fluid-lift culture systems. However, it will be understood that only those cell types which remain relatively strongly attached to culture surfaces during mitosis can be grown with this method. The liquid movement caused by the gas bubbles can cause greater shear forces thus damages or even kills the cells. The instant invention can also be used to increase the surface area of simple cell culture devices such as Petri dishes, wells and tubes.

The novel growth surface of the instant invention can also be used with a bioreactor and/or novel growth surface that provides a periodic movement of media such that the growth surface is alternately submerged and intermittently exposed to air. Such a system is described in U.S. application Ser. No. 10/245,254 incorporated herein in its entirety. Further, the novel growth surface of the present invention can be used with a highly efficient cell-cultivating device such as that described in U.S. Pat. No. 6,323,022, which teaches a cell-cultivating device that includes a plurality of culture tanks and a driving device wherein the culture tanks communicate with each other and have culture medium inside. In this system, the driving means forces the culture medium to flow between the culture tanks so as to vertically oscillate medium level in the culture tanks.

It will be appreciated that the growth surface of the instant invention can be utilized to produce and harvest any type of cellular product such as but not limited to protein pharmaceuticals, cytokines, interferons, antibodies, hormones, growth factors, insulins, viral products, vaccines, nucleic acids, and enzymes. Further, it will be understood that the instant invention can be utilized to grow and/or maintain specific types of cells, such as cells, tissues or organs for transplantation. One skilled in the art will also appreciate that the novel growth surface according to the instant invention can be used to engineer tissues for example, skin or a tissue graft with stem cells.

The instant invention can also be used to generate tissues and organs. It will be appreciated by one of ordinary skill in the art that organ and tissue generation from stem cells and their subsequent transplantation provide promising treatments for a number of ailments, such as diabetes, Parkinson's disease, liver disease, heart disease and autoimmune diseases. Stem cells can be used to generate tissues that are not rejected by the host due to immune incompatibilities, such is the case with current methods of donor tissue procedures. It will be appreciated that by generating tissues (or organs) from a patient's own stem cells, or by genetically altering heterologous cells so that the recipient immune system does not recognize them as foreign may be practiced in accordance with the growth surface of the present invention. Transplant tissues formed on the novel growth surfaces of the present invention can be generated to provide transplantable tissues for resolving various ailments.

It will be appreciated that any type of tissue that can be generated in the art using other methods can also be generated using the growth surface and methods of the instant invention albeit, in a more efficient, effective, and rapid manner. Any form, shape, or size of the growth surface of the instant invention to facilitate the generation of a tissue is contemplated. For example, a section of skin tissue could be generated using the folded, creased, pleated, bent or deformed growth surface according to the instant invention.

One of ordinary skill in the art will appreciate that the success of a cell culture depends on following the correct procedures for culturing cells. Further, the exact procedures used can be different for each type of cell grown. For example, cell growth properties such as the rate and strength of attachment to culture surfaces can be considered when selecting conditions of inoculation and culture maintenance. Also, other parameters such as the size of the inoculum, the condition of the cells used to inoculate, whether the carriers were equilibrated prior to inoculation, initial stirring, media replenishment, and waste removal can all affect the yield of cells in a culture and thus, the yield of cellular products.

In yet another embodiment, the present invention teaches a method of manufacturing a growth surface for eukaryotic and/or prokaryotic cells comprising the steps of treating a surface to encourage and facilitate cell attachment, providing a flat surface, folding the flat surface in a shape that maximizes surface area, cell attachment, cell adhesion, cell growth, and cell proliferation, forming the flat surface into any geometric shape of desire and disposing the growth surface in any conventional cell cultivating device. Further, the method of manufacturing a growth surface may optionally include trimming and/or bending and/or deforming and/or folding and/or creasing at least one end of the growth surface with any angle such that when medium flows through the growth medium and to the end of the growth surface, a current and/or a turbulence is created that further facilitates the mixing of medium and reattachment of the dislodged cells.

One of ordinary skill in the art will understand the methods of treating a surface to encourage and facilitate cell attachment includes coating the growth surface with proteins and/or any other biological or chemical substance to enhance the cell adhesion or selectively enhance attachment of particular types of cells.

In the present invention, the growth surface can be made from any material that is flexible, yet sturdy and capable of maintaining any configuration given. The materials mentioned above are formed into a substantially flat growth surface. The growth surface is then folded, bent, pleated, creased, deformed or otherwise in order to form a precursor that gives the growth surface a three-dimensional configuration of a growth surface that maximizes surface area, cell attachment, cell growth and cell proliferation. This can be done either manually or by any conventional method. Finally, the growth surface is then disposed in any conventional cell cultivating device.

The method of manufacturing a growth surface may optionally include trimming the growth surface to have any edge with any angle to facilitate fluid and/or culture medium movement hence, results in current or turbulence. The end of the growth surface can be optimally turned or bent or folded in any direction away from the ends of the growth surface so that as nutrients and/or culture medium flows through the medium it will create a turbulence, or a current or a ripple in the liquid to facilitate mixing hence even distribution of nutrients and aeration to all cells on the growth surface. The turbulence can mobilize and facilitate cells that are not yet adhered to or dislodged from the growth surface a second chance to re-attach themselves to the growth surface. The trimming or bending or deforming or creasing of the growth surface ends can be done either manually by hand or by any conventional device.

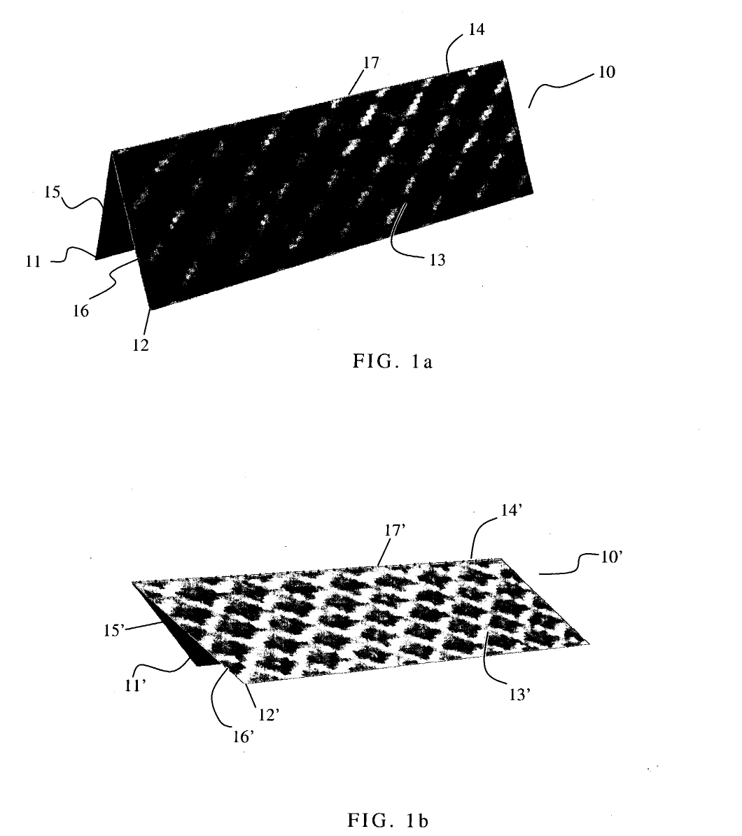

Reference is now made to the figures by way of examples and they are by no way limiting the scope of the present invention. FIGS. 1a and 1b depict a growth surface 10 and 10′ of the first embodiment of the present invention. FIG. 1a depicts a growth surface 10 having surfaces 13, 14, 15 and 16 which has at least a first end 11 and a second end 12. Growth surface 10 has at least one bend or a fold or a crease or a deformation or a precursor that gives the growth surface 10 a three-dimensional configuration, i.e. a V-shape. The bend or the crease or the deformation 17 creates a three-dimensional cavity to house the cells and to prevent surfaces 15 and 16 from overlapping with each other. Surfaces 13, 14, 15 and 16 each is capable of facilitating cell adhesion and promotes cell growth and/or proliferation. The first end 11 and the second end 12 can optimally be trimmed to have an edge with any angle. First end 11 and second end 12 can also be optionally turned in any direction away from surfaces 15 and 16 thus, as nutrients and/or culture medium flows through ends 11 and 12 it will create a turbulence, or a current or a ripple in the liquid to facilitate mixing. This current and/or ripple and/or turbulence facilitates even distribution of nutrients and aeration to all cells on surfaces 13, 14, 15 and 16. The turbulence in addition to mixing also mobilizes cells that are not yet adhered to surfaces 13, 14, 15 and 16 or any dislodged cells a second chance to adhere to the growth surface. Growth surface is made from any material that is flexible, yet sturdy and capable of maintaining any configuration. The growth surface can be made from any polymer such as polypropylene and/or polyethylene or resins. The growth surface can also be made from a non-woven fabric made from interconnected pieces of polyethylene terephthalate (PET) fabric or a sheath-core fabric wherein the sheath component is high-density polyethylene (HDPE) and the core component is polyethylene terephthalate (PET). Growth surface 10 can be porous or nonporous or poreless but preferably porous to house the cells. Because the cells are embedded in the porous growth surface 10, the cells are not directly exposed to shearing stress created by air and medium directly during aeration and feeding thus decrease the cells' mortality rates and increased cellular product.

FIG. 1b depicts growth surface 10′ of the first embodiment of the present the present invention with first end 11′ and second end 12′ trimmed to have any edge with any angle, preferably not a right angle, thus, as nutrients and/or culture medium flows through the growth surface 10′, the medium it will create a turbulence, or a current or a ripple in the liquid to facilitate mixing hence even distribution of nutrients and aeration to all cells on surfaces 13′, 14′, 15′ and 16′.

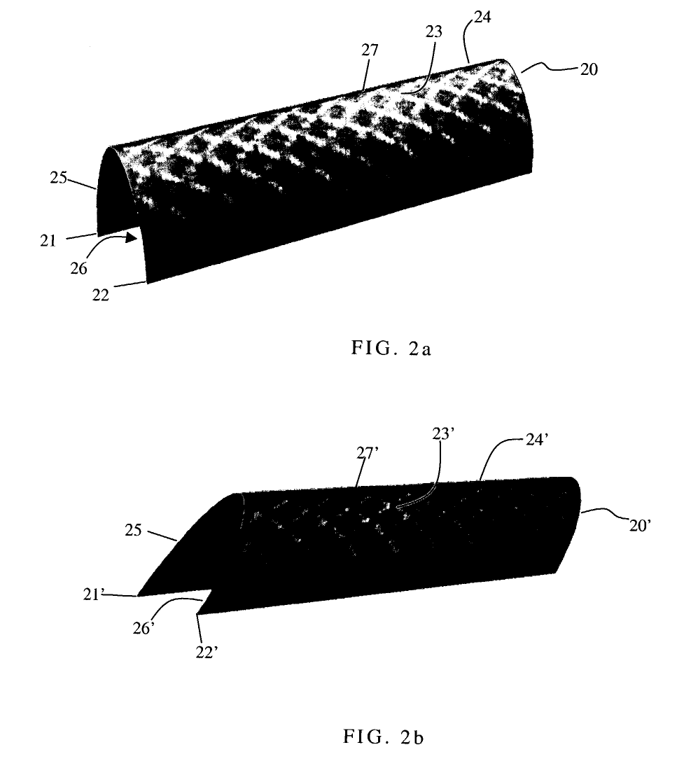

FIGS. 2a and 2b depict growth surface 20 and 20′ of the second embodiment of the present invention. FIG. 2a depicts a growth surface 20 having surfaces 23, 24, 25 and 26 which has at least a first end 21 and a second end 22. Growth surface 20 has at least one bend or a fold or a crease or a deformation or a precursor that gives the growth surface 20 a three-dimensional configuration, i.e. a U-shape. The bend or the crease or the deformation 27 creates a three-dimensional cavity to house the cells and also to prevent surfaces 25 and 26 from overlapping each other thus causing cell death. Surfaces 23, 24, 25, 26 each is capable of facilitating cell adhesion and promotes cell growth and/or proliferation. The first end 21 and the second end 22 can optionally be trimmed to have any edge with any angle. First end 21 and second end 22 can also be optionally turned in any direction away from ends 21 and 22 or growth surface 20 thus, as nutrients and/or culture medium flows through ends 21 and 22, the medium will create a turbulence, or a current or a ripple in the liquid to facilitate mixing and reattachment of the unattached and/or dislodged cells a second chance to attach themselves to the growth surface. This current also creates and causes and facilitates the even distribution of nutrients and aeration to all cells on surfaces 23, 24, 25 and 26.

FIG. 2b depicts growth surface 20′ of the second embodiment of the present the present invention with first end 21′ and second end 22′ trimmed to have any edge with any angle thus, as nutrients and/or culture medium flows through ends 21′ and 22′ it will create a turbulence, or a current or a ripple in the liquid to facilitate mixing hence even distribution of nutrients and aeration to all cells on surfaces 23′, 24′, 25′ and 26′.

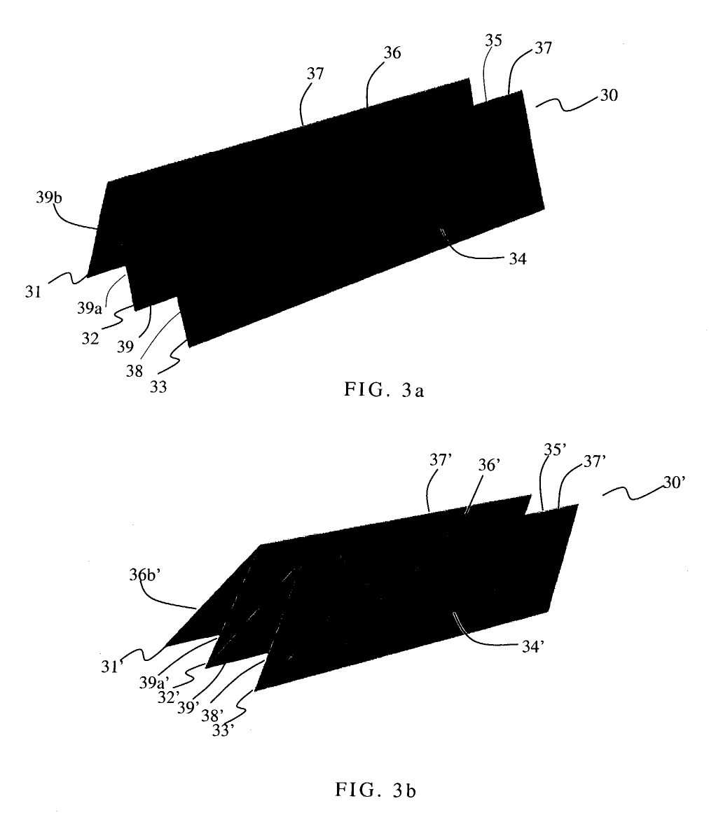

FIGS. 3a and 3b depict growth surface 30 and 30′ of the third embodiment of the present invention. FIG. 3a depicts a growth surface 30 having surfaces 34, 35, 36, 37, 38, 39, 39a, and 39b which has a first end 31, a second end 32 and a third end 33. Growth surface 30 has at least one but preferably two bends or folds or creases or deformations or precursors that give the growth surface 30 at least one three-dimensional configuration, i.e. a W-shape. The bend or the crease or the deformation 37 creates a three-dimensional cavity to house the cells. Surfaces 34, 35, 36, 37, 38, 39, 39a, and 39b each is capable of facilitating cell adhesion and promotes cell growth and/or proliferation. The first end 31, the second end 32 and third end 33 can optionally be trimmed to have any edge with any angle. First end 31, second end 32 and third end 33 can also be optionally turned in any direction away from ends 31, 32 and 33 and surfaces 34, 35, 36, 37, 38, 39, 39a and 39b thus, as nutrients and/or culture medium flows through ends 31, 32, and 33 the medium will create a turbulence, or a current or a ripple in the liquid to facilitate mixing hence even distribution of nutrients and aeration to all cells on surfaces 34, 35, 36, 37, 38, 39, 39a, and 39b as well as providing the unattached and/or dislodged cells a second chance to adhere to growth surfaces 34, 35, 36, 37, 38, 39, 39a and 39b.

FIG. 3b depicts growth surface 30′ of the third embodiment of the present the present invention with first end 31′, second end 32′ and third end 33′ trimmed to have any edge with any angle but a right angle thus, as nutrients and/or culture medium flows through ends 31, 32 and 33 the medium will create a turbulence, or a current or a ripple in the liquid to facilitate mixing hence even distribution of nutrients and aeration to all cells on surfaces 34′, 35′, 36′, 37′, 38′, 39′, 39a′, and 39b′.

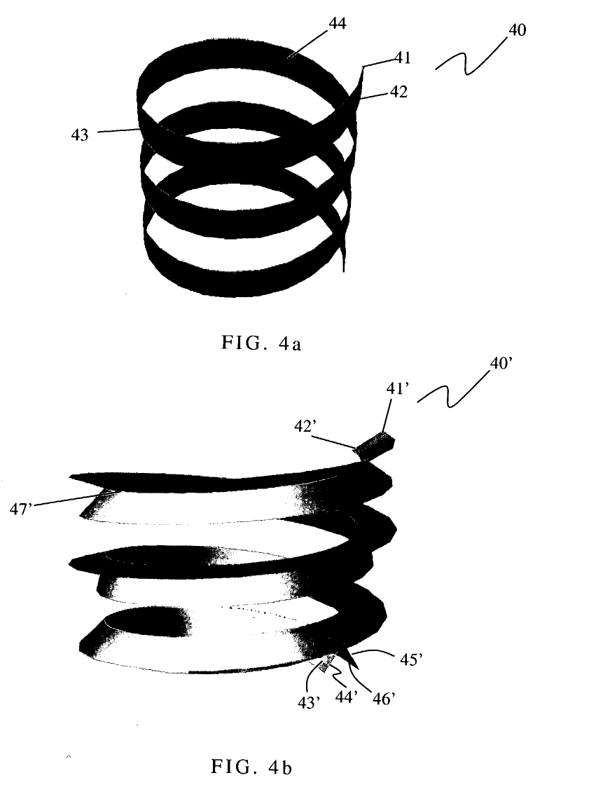

FIGS. 4a and 4b depict growth surface 40 and 40′ of the fourth embodiment of the present invention. FIG. 4a depicts a growth surface 40 having surfaces 43 and 44 which has a first end 41 and a second end 42. Growth surface 40 has two surfaces 43 and 44 and shaped like a ribbon. Surfaces 43 and 44 each is capable of facilitating cell adhesion and promotes cell growth and/or proliferation. The first end 41 and the second end 42 can optionally and preferably be trimmed to have any edge with any angle but a right angle. First end 41 and second end 42 can also be optionally turned in any direction away from ends 41 and 42 thus, as nutrients and/or culture medium flows through ends 41 and 42, the medium will create a turbulence, or a current or a ripple in the liquid to facilitate mixing hence even distribution of nutrients and aeration to all cells on surfaces 43 and 44.

FIG. 4b depicts growth surface 40′ of the forth embodiment of the present invention. FIG. 4b depicts a growth surface 40′ having surfaces 43′, 44′, 45′, and 46′ which has a first end 41′ and a second end 42′. Growth surface 40′ has at least one bend or a fold or a crease or a deformation or a precursor that gives the growth surface 40′ a three-dimensional configuration. The bend or the crease or the deformation 47′ creates a three-dimensional cavity to house the cells and also to prevent growth surfaces 44′ and 46′ from overlapping each other thus injuring and/or killing the cells. Surfaces 43′, 44′, 45′, and 46′ each is capable of facilitating cell adhesion and promotes cell growth and/or proliferation. The first end 41′ and the second end 42′ can optionally be trimmed to have any edge with any angle but a right angle. First end 41′ and second end 42′ can also be optionally turned in any direction away from ends 41′ and 42′ thus, as nutrients and/or culture medium flows through the ends 41′ and 42′ the medium will create a turbulence, or a current or a ripple in the liquid to facilitate mixing hence even distribution of nutrients and aeration to all cells on surfaces 43′, 44′, 45′, and 46′.

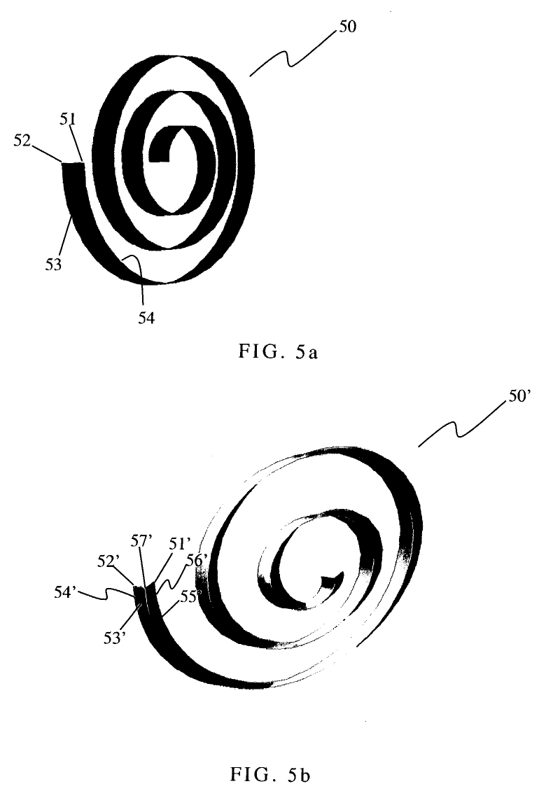

FIGS. 5a and 5b depict growth surface 50 and 50′ of the fifth embodiment of the present invention. FIG. 5a depicts a growth surface 50 having surfaces 53 and 54 which has a first end 51 and a second end 52. Growth surface 50 does not have a bend or a fold or a crease or a deformation or a precursor however it is curved to created a three-dimensional configuration, i.e. a spiral-shape. This three-dimensional configure creates a cavity to house and shelter cells. Surfaces 53 and 54 each is capable of facilitating cell adhesion and promotes cell growth and/or proliferation. The first end 51 and the second end 52 can optionally be trimmed to have any edge with any angle but a right angle. First end 51 and second end 52 can also be optionally turned in any direction away from ends 51 and 52 thus, as nutrients and/or culture medium flows through ends 51 and 52 the medium will create a turbulence, or a current or a ripple in the liquid to facilitate mixing hence even distribution of nutrients and aeration to all cells on surfaces 53 and 54.

FIG. 5b depicts growth surface 50′ of the fifth embodiment of the present invention. FIG. 5b depict a growth surface 50′ having surfaces 53′, 54′, 55′, and 56′ which has at least a first end 51′ and a second end 52′. Growth surface 50′ has at least one bend or a fold or a crease or a deformation or a precursor that gives the growth surface 50′ a three-dimensional configuration, i.e. a three-dimensional spiral. The bend or the crease or the deformation 57′ creates a three-dimensional cavity to house the cells. Surfaces 53′, 54′, 55′, and 56′ each is capable of facilitating cell adhesion and promotes cell growth and/or proliferation. The first end 51′ and the second end 52′ can optionally be trimmed to have any edge with any angle. First end 51′ and second end 52′ can also be optionally turned in any direction away from ends 51′ and 52′ thus, as nutrients and/or culture medium flows through the ends 51′ and 52′ the medium will create a turbulence, or a current or a ripple in the liquid to facilitate mixing hence even distribution of nutrients and aeration to all cells on surfaces 53′, 54′, 55′, and 56′.

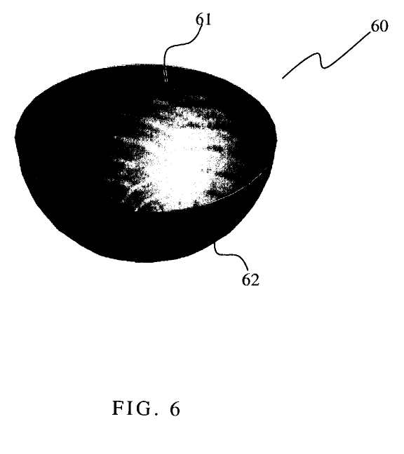

FIG. 6 depicts a growth surface of the sixth embodiment of the present invention. FIG. 6 depict a growth surface 60 having surfaces 61 and 62. Growth surface 60 does not have a bend or a fold or a crease however, it has a deformation that gives the growth surface 60 a three-dimensional bowl-shape structure. The deformation creates a three-dimensional cavity to house the cells. Surfaces 61 and 62 each is capable of facilitating cell adhesion and promotes cell growth and/or proliferation as well as a second chance for unattached and dislodged cells to re-attach themselves to the growth surface.

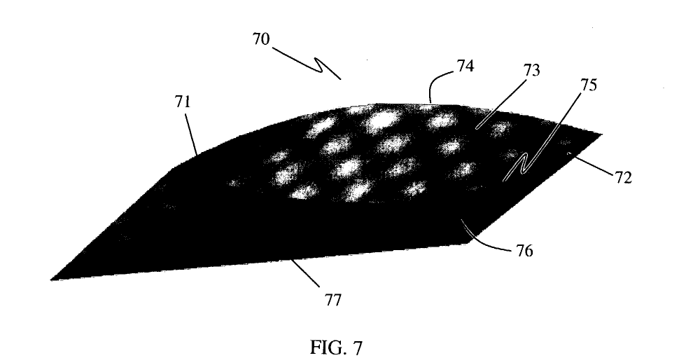

FIG. 7 depicts a growth surface of the seventh embodiment of the present invention. FIG. 7 depicts a growth surface 70 having surfaces 73, 74, 75 and 76 which has at least a first end 71 and a second end 72. Growth surface 70 has at least one bend or a fold or a crease or a deformation or a precursor that gives the growth surface 70 a three-dimensional configuration, i.e. a boat-shape. The bend or the crease or the deformation 77 creates a three-dimensional cavity to house the cells and also to prevent surfaces from overlapping each other thus causing cell death. Surfaces 73, 74, 75 and 76 each is capable of facilitating cell adhesion and promotes cell growth and/or proliferation. The first end 71 and the second end 72 can optionally be trimmed to have any edge with any angle. First end 71 and second end 72 can also be optionally turned in any direction away from ends 71 and 72 or growth surface 70 thus, as nutrients and/or culture medium flows through ends 71 and 72, the medium will create a turbulence, or a current or a ripple in the liquid to facilitate mixing and reattachment of the unattached and/or dislodged cells a second chance to attach themselves to the growth surface. This current also creates and causes and facilitates the even distribution of nutrients and aeration to all cells on surfaces 73, 74, 75 and 76.

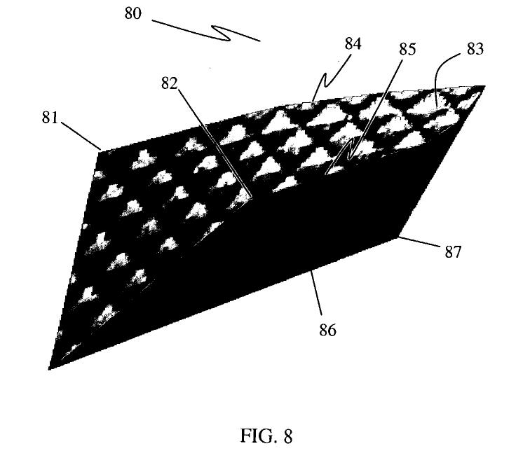

FIG. 8 depict growth surface 80 of the second embodiment of the present invention. FIG. 8 depicts a growth surface 80 having surfaces 83, 84, 85 and 86 which has at least a first end 81 and a second end 82. Growth surface 80 has at least one bend or a fold or a crease or a deformation or a precursor that gives the growth surface 80 a three-dimensional configuration, i.e. a shovel-shape. The bend or the crease or the deformation 87 creates a three-dimensional cavity to house the cells and also to prevent surfaces from overlapping each other thus causing cell death. Surfaces 83, 84, 85 and 86 each is capable of facilitating cell adhesion and promotes cell growth and/or proliferation. The first end 81 and the second end 82 can optionally be trimmed to have any edge with any angle. First end 81 and second end 82 can also be optionally turned in any direction away from ends 81 and 82 or growth surface 80 thus, as nutrients and/or culture medium flows through ends 81 and 82, the medium will create a turbulence, or a current or a ripple in the liquid to facilitate mixing and reattachment of the unattached and/or dislodged cells a second chance to attach themselves to the growth surface. This current also creates and causes and facilitates the even distribution of nutrients and aeration to all cells on surfaces 83, 84, 85 and 86.

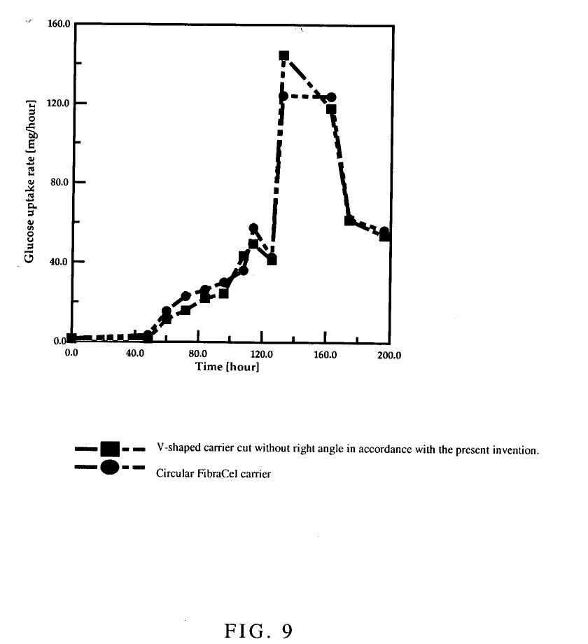

FIG. 9 depicts a comparison for an overall glucose consumption rate per hour by cells over time between cells plated on the growth surface illustrated in FIG. 1b of the present invention and the conventional FIBRA-CEL® disk-shaped carriers. The glucose consumption rate is proportional to cell growth.

The square boxes on the graph illustrates the glucose consumption rate per hour when cells are grown on the V-shaped growth surface of the present invention. The filled-in ovals on the graph depict the glucose consumption rate per hour of cells plated on conventional FIBRA-CEL® disk-shaped carriers. The glucose consumption rate is proportional to cell growth.

The graph of FIG. 9 shows that the glucose uptake rate for cells grown on the V-shaped growth surfaces in accordance with the present invention is significantly higher than cells grown on the conventional FIBRA-CEL® disk-shaped carriers particularly 120 hours after cell inoculation. Thus the V-shaped growth surface of the present invention significantly improves cell attachment, cell growth and cell proliferation thus, increased cellular products. For instance, at approximately 130 hours the cells' glucose uptake rate is approximately 145 mg/hour whereas the cells plated on the conventional FIBRA-CEL® disk-shaped carriers has only 120 mg/hour of glucose consumption rate. The glucose uptake rate is significantly higher for cells raised on V-shaped growth surface of the present invention as compares to the cells plated on conventional FIBRA-CEL® disk-shaped carriers. Further, the graph illustrates that the maximum surface area of the V-shaped growth surface is significantly higher than that of the conventional FIBRA-CEL® disk-shaped carriers and thus, cells plated in the V-shaped growth surface will have a higher density. The experimental data shows that cells plated on the V-shaped growth surface of the present invention has surprisingly high glucose intake over time.

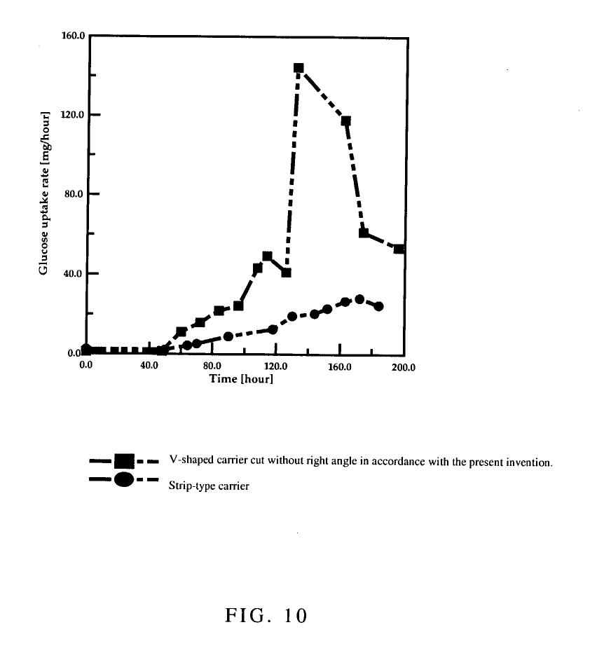

FIG. 10 shows a comparison for an overall glucose consumption rate per hour by cells over time between cells adhered to the growth surface shown in FIG. 1b of the present invention and cells adhered to a conventional strip-type carrier without any three-dimensional structure.

The square boxes on the graph illustrates the glucose consumption rate per hour when cells are plated on the V-shaped growth surface of the present invention. The filled-in ovals on the graph depicts the glucose consumption rate per hour of a cells grown on the conventional strip-type carrier without any three-dimensional structure. The glucose consumption rate is proportional to cell growth.

The graph of FIG. 10 shows that the glucose uptake rate for cell plated on the V-shaped growth surface in accordance with the present invention is significantly higher than cells grown on a conventional two-dimensional strip-type carrier that does not have any three-dimensional structure. Thus the V-shaped growth surface of the present invention significantly improves cell attachment, cell growth and cell proliferation thus, increased cellular production. For instance, at approximately 143 hours the cells' glucose uptake rate is 145 mg/hour whereas the cells plated on the conventional strip-type carrier without any three-dimensional structure is only 20 mg/hour. The glucose uptake rate is approximately 7 times higher for the V-shaped growth surface of the present invention. The experimental data clearly supports the surprising results caused by the unique growth surface claimed and disclosed in the present invention.

### EXAMPLES

### Example 1

This example compares the cell cultivation efficiencies of the V-shaped growth surface having at least one non-right angle turning point disposed at the first end and the second end of the growth surface (shown in FIG. 1b) and the commercialized FIBRA-CEL® disk-shaped carriers.

First, the growth surfaces of the present invention were trimmed and folded into a V-shaped growth surface with a non-right-angle-shaped turning point. The V-shape growth surface is soaked in 99.5% alcohol for one hour and then rinsed twice with lab-grade water. After drying 7.0 grams of the growth surfaces at 28° C., the growth surfaces were placed in a high-efficiency bioreactor. (See U.S. Pat. No. 6,323,022). The bioreactor was prepared by filling the reactor with PBS solution and then completing a 30-minute high temperature sterilization. After complete sterilization, the PBS solution was replaced with culture medium (DMEM with 10% calf serum) in the reactor. CHO-dhfr cells (Chinese hamster ovary cells) with a density of 2.5×10⁵ cells/cm³ were introduced into the bioreactor. The cells in the bioreactor were incubated for sufficient time for cells to attach onto the growth surfaces wherein the amount of time depends on the type of cell and the surface properties of the growth surface. Subsequently, the cells were grown under cultivation conditions. Once the density of the residual glucose reached below 1200-1500 mg/L, a semi-continuous mode of cultivation was adopted. Since the uptake of glucose is proportional to the number of cells, cell growth was indirectly monitored by measuring the glucose uptake rate (GUR).

The results, as shown in FIG. 9 (GUR vs. cultivation time), demonstrate that the growth surface of the present invention is superior to that of the FIBRA-CEL® when growth is compared under the same cultivation conditions over a period of 140 hours since the GUR value is significantly higher for cells plated on the V-shaped surfaces of the present invention than cells plated on the FIBRA-CEL® disk-shaped carriers.

### Example 2

This example compares the cell cultivation efficiencies of the V-shaped growth surface having at least one non-right angle turning point disposed at the first end and the second end of the growth surface (shown in FIG. 1b) and the conventional strip-type carriers of the same material.

First, the growth surfaces of the present invention were divided into two groups: growth surfaces folded into a V-shape and an unfolded flat strip-type carriers. The procedure set forth below were implemented for the folded growth surfaces and the non-folded growth surfaces. The growth surfaces were soaked in 99.5% alcohol for one hour and then rinsed twice with lab-grade water. After drying 7.0 grams of the carriers at 28° C., the carriers were placed in a high-efficiency bioreactor (See U.S. Pat. No. 6,323,022). The bioreactor was prepared by filling it with PBS solution and then completing a 30-minute high temperature sterilization. The PBS solution was replaced with culture medium (DMEM with 10% calf serum) in the reactor.

CHO-dhfr cells (Chinese hamster ovary cells) with a density of 2.5×10⁵ cells/cm³ were introduced into the bioreactor. The cells in the bioreactor were incubated for sufficient time for cells to attach onto the growth surfaces wherein the amount of time depends on the type of cell and the surface properties of the growth surface. Subsequently, the cells were grown under cultivation conditions. Once the density of the residual glucose reached below 1200-1500 mg/L, a semi-continuous mode of cultivation was adopted. Since the uptake of glucose is proportional to the number of cells, cell growth was indirectly monitored by measuring the glucose uptake rate (GUR).

As shown in FIG. 10, the GUR value obtained when cells are plated on the V-shaped growth surfaces of the present invention is significantly higher than the value from cells plated on the strip-type carriers under the same cultivating conditions when the cultivating time is only 50 hours. Therefore, the V-shaped growth surface of the present invention is superior to conventional cell cultivating carriers.

As shown above, the growth surface and its manufacturing process provided by the present invention effectively minimizes the problem of reduced space for cell attachment due to significant overlapping in the cultivating tank associated with traditional flat strip carriers. Furthermore, the growth surface provides branch-like channels for the effective transportation of nutrients, air and cell wastes to and from cells on each growth surface.

### Example 3

The growth surface of the present invention will be used to produce murine leukemia virus proviral DNA by culturing the mouse bone marrow cell line JLS-V9 cells infected with Moloney leukemia virus (M-MuLV).

First, the growth surface of the present invention will be provided. The growth surface according to the present invention can be formed in any desired shape, preferably creating a three-dimensional cavity such as W-shaped, V-shaped, ribbon-shaped, spiral-shaped, bowl-shaped, boat-shaped, shovel-shaped, or dust-pan shaped growth surface. The size growth surface will be on the order of about 1 cm to about 4 cm². A single growth surface or a plurality of growth surfaces can be used for the purposes of this Example.

The growth surface will be pretreated by soaking in 99.5% alcohol for one hour, rinsing twice with lab-grade water and then allowing it to dry. The growth surface will be placed into a traditional bioreactor, such as a roller bottle, followed by the addition of a buffer solution to submerge the growth surfaces. The roller bottle and contents will then be sterilized using high heat for 30 minutes.

Following sterilization, the buffer solution will be replaced with culture medium, such as Dulbecco's Modification of Eagle's Medium (DMEM) containing 10% fetal bovine serum and mixed at 37° C. at a 5% CO₂ environment until the medium is equilibrated. The growth surfaces will then be inoculated with approximately 1.5×10⁷ JLS-V9 cells and grown for a sufficient number of hours to allow the cells to proliferate.

The spent medium will then be removed and the M-MuLV virus will be inoculated into the roller bottles. Shortly thereafter, the culture will be fed with fresh medium. Eight to sixteen hours later the culture will be extracted for viral DNA. To extract the DNA, the culture will be washed with fresh buffer and the cells will be lysed with a solution containing the detergent sodium dodecylsulfate. High molecular weight DNA will be precipitated by adding salt to one molar, leaving the low molecular weight viral DNA in solution which will then be deproteinized and concentrated for further analysis.

### EXAMPLE 4

The growth surfaces of the present invention can be treated to further encourage cell growth. Cell adhesion factors, such as growth factors, selected protein, amino acids, collagen, antibodies, chemical materials and other such materials can be added to the growth surfaces of the present invention to facilitate cell attachment and spreading. Further, charged molecules can be added to further stabilize cell attachment and growth which provides a more efficacious culture system.

In this example, the preparation of a growth surface of the present invention with modified surface properties will be outlined. The growth surface of the present invention will be prepared using non-amine exchange groups to form a positively charged surface which will facilitate cell attachment and spreading.

In a reaction vessel, 5 milliliters of a saturated aqueous solution of sodium triethyl-(ethyl-bromide)-phophonium (TEP) and 5 milliliters of a 3 molar solution of sodium hydroxide will be added to about one gram of the growth surfaces of the present invention. The mixture will be reacted at 65° C. for a span of time ranging from about 5 minutes to about 1 hour. The cellular products will be retrieved later for further analysis.

### Example 5

The growth surface of the present invention will be used to culture a genetically engineered line of CHO cells, comprising a stably-integrated and expressible DNA encoding a foreign protein of interest such as human interferon. The recombinant cells will be cultured using the growth surfaces of the present invention and a highly-efficient bioreactor or cell cultivating device such as that described in U.S. application Ser. No. 10/245,254 or those available commercially from Cesco Bioengineering, Inc., such as the BELLOCELL™ or TIDECELL® bioreactor.

First, the growth surface of the present invention is provided. The growth surface according to the present invention can be formed in any desired shape, preferably creating a three-dimensional cavity such as W-shaped, V-shaped, ribbon-shaped, spiral-shaped, bowl-shaped, boat-shaped, shovel-shaped, or dust-pan shaped growth surface. The growth surface in this case is porous and can be made from a non-woven fiber. The growth surface will be on the order of about 1 cm² to about 4 cm². A single growth surface or a plurality of growth surfaces can be used for the purposes of this Example.

The growth surface will be pretreated by soaking them in 99.5% alcohol for one hour, rinsing twice with lab-grade water and then allowing them to dry. The growth surface will then be placed into the highly-efficient cell cultivating device disclosed in U.S. application Ser. No. 10/245,254 or those available commercially from Cesco Bioengineering, Inc., such as the BELLOCELL™ or TIDECELL® bioreactor: Buffer will be added to submerge the growth surface in the cell cultivating device and then the system will be sterilized with heat.

The buffer will be replaced with an appropriate medium, such as DMEM with 10% calf serum. Next, the recombinant cell line will be used to inoculate the growth surface of the present invention with a density of 2.5×10⁵ cells/cm³. Once growth medium and the inoculum are added, the cell-cultivating device will be placed into operation such that the growth surfaces become intermittently and periodically submerged in growth medium and air containing an optimum concentration of oxygen in accordance with the invention of U.S. application Ser. No. 10/245,254 or the commercially available BELLOCELL™ and/or TIDECELL® (Cesco Bioengineering, Inc.).

The movement of the growth medium over, around and through the growth surfaces of the instant invention will create turbulence due to the bends or folds of the growth surfaces and optionally for the trims at the ends of the growth surface or the folding away of these ends which will facilitate the mixing and homogenization of the culture medium, the redistribution and reattachment of dislodged cells, and the oxygenation of the culture. Further, the growth surfaces prepared in accordance with the instant invention will provide channels to promote the flow of growth medium through and around the cells due to the bends or folds of the growth surface. During growth of the culture, the foreign protein of interest will be secreted into the growth medium and will accumulate in concentration over time.

Once an optimum level of cell growth is achieved, e.g., the growth medium becomes exhausted of all nutrients, the cell growth medium will be removed from the cell-cultivating device and replenished with fresh medium. The cell growth medium will contain the recombinant protein of interest, which can be purified according to methods well-known in the art.

What is claimed is:

1. A growth surface comprising:
a growth surface adapted to maximize surface area suitable for cell attachment and cell growth by imparting a geometric configuration to the growth surface.

2. The growth surface of claim 1, wherein the surface is porous.

3. The growth surface of claim 1, wherein the surface is nonporous.

4. The growth surface of claim 1, wherein the surface is a polymer.

5. The growth surface of claim 1, wherein the surface is polyethylene terephthalate or high-density polyethylene.

6. The growth surface of claim 1, wherein the surface is made from a non-woven fabric.

7. The growth surface of claim 6, wherein the non-woven fabric is optionally treated to facilitate cell attachment.

8. The growth surface of claim 6, wherein the non-woven fabric comprises interconnected pieces of polyethylene terephthalate.

9. The growth surface of claim 6, wherein the non-woven fabric comprises a sheath-core.

10. The growth surface of claim 9, wherein the sheath comprises high-density polyethylene and the core comprises polyethylene terephthalate.

11. A method for making a growth surface comprising:
forming a growth surface having a first end and a second end; and
bending and/or folding the growth surface to create additional growth surface.

12. The method of claim 111 further comprising trimming at least the first end of the growth surface to facilitate mixing a growth medium statically.

13. The method of claim 11 further comprising bending and/or folding the first end in an angle to create a fluid current and/or fluid turbulence.

14. The method of claim 11, further comprising the step of treating the growth surface to facilitate cell attachment.

15. The growth surface of claim 1, further comprising a fold or deformation to create at least one three-dimensional cavity.

16. The growth surface of claim 1, wherein the surface is a pellet.

17. The growth surface of claim 1, wherein the surface is a strip.

18. The growth surface of claim 1, wherein the surface is V-shaped.

19. The growth surface of claim 1, wherein the surface is U-shaped.

20. The growth surface of claim 1, wherein the surface is W-shaped.

21. The growth surface of claim 1, wherein the surface is ribbon-shaped.

22. The growth surface of claim 1, wherein the surface is spiral-shaped.

23. The growth surface of claim 1, wherein the surface is bowl-shaped.

24. The growth surface of claim 1, wherein the surface is boat-shaped.

25. The growth surface of claim 1, wherein the surface is shovel-shaped.

26. The growth surface of claim 1, further comprising a fold or deformation to create a turbulence to facilitate even distribution of nutrients and aeration to cells.

27. The growth surface of claim 1, further comprising a fold or deformation to mobilize unattached cells for cell attachment.

28. A growth surface comprising:
a surface to facilitate cell attachment and cell growth, wherein at least one fold or deformation is imparted on the growth surface to create at least one three-dimensional cavity and trimming at least one end of the surface to create a liquid turbulence to facilitate even distribution of nutrients and aeration to cells.

29. A growth surface comprising:
a surface to facilitate cell attachment and cell growth, wherein the growth surface comprises a three-dimensional configuration capable of creating turbulence to facilitate even distribution of nutrients and aeration to cells and mobilizing unattached or dislodged cells for reattachment.

30. The method of claim 11, wherein the surface is V-shaped.

31. The method of claim 11, wherein the surface is U-shaped.

32. The method of claim 11, wherein the surface is W-shaped.

33. The method of claim 11, wherein the surface is ribbon-shaped.

34. The method of claim 11, wherein the surface is spiral-shaped.

35. The method of claim 11, wherein the surface is bowl-shaped.

36. The method of claim 11, wherein the surface is boat-shaped.

37. The method of claim 11, wherein the surface is shovel-shaped.

38. The method of claim 11, wherein bending and/or folding the growth surface creates a three-dimensional structure.

39. The method of claim 11, wherein bending and/or folding the growth surface creates a liquid turbulence to facilitate even distribution of nutrients and aeration to cells, and mobilize unattached or dislodged cells for cell re-attachment.

40. A method for making a growth surface comprising:
forming a growth surface having a first end and a second end;
treating the growth surface to facilitate cell attachment and affinity; bending and/or folding the growth surface to create a three-dimensional structure; and disposing the growth surface within a cultivating device.

41. A method for making a growth surface comprising:
forming a growth surface having a first end and a second end;
bending and/or folding the growth surface to create a three-dimensional structure; and trimming at least the first end to create turbulence to facilitate even distribution of nutrients and aeration to cells.
